(19) United States
(12) Patent Application Publication
(10) Pub. No.: US 2021/0217494 A1
(43) Pub. Date: Jul. 15, 2021

(54) METHOD AND SYSTEM FOR USE IN DIRECT SEQUENCING OF RNA

(71) Applicant: New York Institute of Technology, Old Westbury, NY (US)

(72) Inventors: Shenglong Zhang, Fort Lee, NJ (US); Tom Z. Wang, Brooklyn, NY (US); Tony Z. Jia, Tokyo (JP); Wenjia Li, Scarsdale, NY (US)

(21) Appl. No.: 17/058,165

(22) PCT Filed: May 24, 2019
(86) PCT No.: PCT/US2019/033895

§ 371 Date: Nov. 24, 2020

Related U.S. Application Data
(60) Provisional application No. 62/676,754, filed on May 25, 2018.

(51) Int. Cl. G16B 40/10; G01N 30/72; G01N 30/86

(52) U.S. Cl. CPC G16B 40/10; G01N 30/8675; G01N 30/7233

(57) ABSTRACT

The present disclosure relates generally to systems and methods for determining an order of nucleotides of an RNA molecule. The method includes receiving liquid chromatography-mass-spectrometry (LC-MS) data of an RNA sample, filtering the LC-MS data based on mass, the filtering including removing masses smaller than a predetermined size, analyzing the filtered LC-MS data, to determine a plurality of RNA sequences, and reading-out an RNA sequence after determining no remaining valid nucleotides in the remaining LC-MS data. Analyzing the filtered LC-MS data includes determining a mass difference between at least two adjacent ladder fragments, and determining whether the mass difference is equal to a canonical nucleotide, or a modified nucleotide. The LC-MS data including a mass, retention time (RT), and volume. The RNA sequence including a sequence of each identified canonical nucleotide and any identified modified nucleotides.

### CROSS-REFERENCE TO RELATED APPLICATION

This application claims benefit and priority to U.S. Provisional Application No. 62/676,754, filed May 25, 2018, which is incorporated herein by reference in its entirety.

### TECHNICAL FIELD

The present disclosure relates generally to novel algorithms developed for liquid chromatography-mass-spectrometry (LC-MS) based RNA sequencing techniques based on end-labeling of RNA to be sequenced and the fragmented ladders of RNA that cover the complete suite of ladder fragments from first ribonucleotide to the final one. The algorithms simultaneously read a target RNA sequence with single nucleotide resolution and determine the presence, type, location, and quantity of a wide spectrum of target RNA modifications. The disclosed algorithms introduce computational simulations resulting in reciprocal verification between experimental data and simulated data. The simulation provides a means for sequencing RNA molecules of increased length as well as RNA samples with increased strands and population diversity.

### BACKGROUND

Mass spectrometry (MS) is a tool for studying protein modifications, where peptide fragmentation produces “ladders” that reveal the identity and position of various amino acid modifications. As of yet, a similar approach is not yet feasible for nucleic acids, because in situ fragmentation techniques providing satisfactory sequence coverage do not exist. Aberrant nucleic acid modifications, especially methylations and pseudouridylations in RNA, have been correlated to the development of major diseases like breast cancer, type-2 diabetes, and obesity, each of which affects millions of people around of the world. Despite their significance, the available tools to reliably identify, locate, and quantify modifications in RNA are very limited.

Accordingly, new methods are needed to facilitate the efficient sequencing of RNA molecules.

### SUMMARY

To enable automated direct sequencing of RNA, algorithms with improved accuracy are desired, given that LC/MS data contains data from multiple-cut RNA fragments, making it difficult to analyze, especially for the sequences to be generated from the lower mass regions where smaller degraded RNA fragments are located. The present disclosure relates to development of an algorithm for use with mass RNA laddering sequencing methods.

In accordance with aspects of the present disclosure, a computer implemented method for determining an order of nucleotides of an RNA molecule is presented. The method includes: receiving liquid chromatography-mass-spectrometry (LC-MS) data of an RNA sample, filtering the LC-MS data based on mass, analyzing the filtered LC-MS data to determine an RNA sequence, and reading-out an RNA sequence as a sequence read based on determining no remaining valid nucleotides in the remaining LC-MS data. The RNA sequence includes a sequence order of each identified canonical nucleotide and any identified modified nucleotides. The LC-MS data including a mass, retention time (RT), volume, and quality score (QS). The filtering including removing masses smaller than a predetermined size. The sequencing includes: determining a mass difference between at least two adjacent ladder fragments, and determining whether the mass difference is equal to at least one of a canonical nucleotide, or a modified nucleotide.

In an aspect of the present disclosure, the method may further include: determining whether there are any gaps in the sequenced LC-MS data, determining whether there are any remaining RNA fragment that did not yield a valid nucleotide based on the gaps, performing a hierarchical clustering algorithm on the compounds to identify possible nucleotides from their related mass-adducts, determining the mass of an RNA fragment for each cluster, based on item-wise comparison between the identified mass-adducts and the cluster of masses, predicting a ladder fragment based on the determined mass for each cluster, reading-out an RNA sequence based on the predicted ladder fragment, and reporting the RNA sequence. The hierarchical clustering algorithm includes: determining a distance metric based on a mass as well as RT for the RNA fragment; and grouping RNA fragment, into a cluster of masses, based on their mass relationship, such that each fragment includes possible mass-adducts of a true ladder fragment. The RNA sequence selected to report out can include the nucleotide identified form any mass-adducts.

In another aspect of the present disclosure, a length of the RNA molecule is more than 20 nucleotides.

In an aspect of the present disclosure, one or more RNA molecules are present in the RNA sample to be sequenced.

In yet another aspect of the present disclosure, the RNA sample includes a purified RNA sample.

In a further aspect of the present disclosure, the RNA sample includes a therapeutic RNA molecule.

In an aspect of the present disclosure, the RNA sequence is determined by correlation of MS data output with a mass of known ribonucleotides.

In a further aspect of the present disclosure, including determining a type, location, and quantity of modified ribonucleotides based on correlating mass-spectrometry (MS) data output with a mass of known modified ribonucleotides.

In yet another aspect of the present disclosure, the sequencing of the filtered LC-MS data is based on a unique property of the RNA fragment. In a further aspect of the present disclosure, the unique property of an RNA fragment includes at least one of electronic or optical signature signals.

In accordance with aspects of the present disclosure, a system for determining an order of nucleotides of an RNA molecule is presented. The system includes a processor and a memory. The memory stores instructions which, when executed by the one or more processors, cause the system to: receive liquid chromatography-mass-spectrometry (LC-MS) data of an RNA sample, the LC-MS data including a mass, retention time (RT), volume, and quality score (QS); filter the LC-MS data based on mass, the filtering including removing masses smaller than a predetermined size; analyzing the filtered LC-MS data, to determine a plurality of RNA sequences, and reading-out an RNA sequence as a sequence read after determining no remaining valid nucleotides in the remaining LC-MS data. The RNA sequence including a sequence of each identified canonical nucleotide and any identified modified nucleotides. Analyzing the filtered LC-MS data includes: determining a mass difference between at least two adjacent ladder fragments; and determining whether the mass difference is equal to at least one of: a canonical nucleotide, or a modified nucleotide.

In accordance with aspects of the present disclosure, a computer implemented method for determining an order of nucleotides of an RNA molecule is presented. The method includes accessing liquid chromatography-mass-spectrometry (LC-MS) data of an RNA sample, the RNA sample including an RNA ladder fragment; accessing a database including theoretical mass, calculated from chemical formula, of all known ribonucleotides including those with modifications to the base; performing anchor-based sub-setting on the LC-MS data, the anchor based sub-setting including selecting a data zone; performing base calling on the selected subset of LC-MS data to generate a dataset of tuples; building trajectories linking tuples in the dataset to generate a draft read of the RNA ladder fragment; and performing a draft read strategy.

In yet a further aspect of the present disclosure, the draft read strategy includes scoring based on at least one of read length, average volume, average QS, or average parts per million (PPM).

In yet another aspect of the present disclosure, PPM is determined as:

$$PPM = \frac{M a s {s}_{experimental} - {Mass}_{theoretical}}{M a s {s}_{theoretical}} \times 1 {0}^{6} ,$$

wherein: Massₑₓₚₑᵣᵢₘₑₙₜₐₗ is an experimental mass corresponding to a molecular tag, and Mass_{theoretical} is the theoretical mass.

In a further aspect of the present disclosure, average PPM is the sum of all PPM values associated with data points contained in a draft read divided by read length.

In yet a further aspect of the present disclosure, building trajectories further includes performing a Depth First Search (DFS) algorithm to ensure that all possible draft reads will be found from the LC-MS data.

In yet another aspect of the present disclosure, the method further includes biochemical labeling of the RNA samples.

In a further aspect of the present disclosure, the draft read strategy includes a global hierarchical ranking strategy.

In an aspect of the present disclosure, the draft read strategy includes a local best score strategy. In another aspect of the present disclosure, the method further includes performing an alignment/assembly algorithm configured to assemble a complete RNA sequence from different fragments of the RNA molecule.

Further details and aspects of exemplary embodiments of the disclosure are described in more detail below with reference to the appended figures. Any of the above aspects and embodiments of the disclosure may be combined without departing from the scope of the disclosure.

### BRIEF DESCRIPTION OF THE DRAWINGS

Various embodiment of the present methods for RNA sequencing and algorithm are described herein with reference to the drawings wherein:

FIG. 1 shows flowchart for the sequencing workflow of the algorithm, in accordance with the present disclosure;

FIG. 2 demonstrates algorithm for base-matching based on mass differences, in accordance with the present disclosure;

FIG. 3 shows formula to determine the mass of ladder fragments obscured by mass-adducts, in accordance with the present disclosure;

FIG. 4 demonstrates computational simulation of the simultaneous base-calling of 3′-mass ladder fragments of three homopolymers, in accordance with the present disclosure;

FIG. 5 demonstrates direct LC-MS sequencing of a 20-nt RNA using the computational algorithm defined by their mass, chromatographic RT and abundance, with 5′-biotin labeling but no bead separation, in accordance with the present disclosure;

FIG. 6 shows the known masses for modified ribonucleotides, in accordance with the present disclosure;

FIG. 7 shows the work flow for 2-Dimensional mass-retention time-based direct sequencing of RNA, in accordance with the present disclosure;

FIG. 8 is a flowchart of a method for determining the order of nucleotides of an RNA molecule in accordance with the disclosure;

FIG. 9 shows the workflow of data analysis using the global hierarchical ranking algorithm, in accordance with the present disclosure;

FIG. 10 shows the workflow of data analysis using the local best score algorithm, in accordance with the present disclosure;

FIG. 11A shows generation of three major fragments by RNase T1 digestion of tRNA detected by LC/MS, Fragment I, II, and III, in accordance with the present disclosure;

FIG. 11B shows selection of data zones in the 2-D RT versus mass plot of test tRNA sequencing output dataset, in accordance with the present disclosure;

FIG. 12 shows pseudo-code of base calling, in accordance with the present disclosure;

FIG. 13 shows pseudo-code/work flow of sequence generation by building trajectories, in accordance with the present disclosure;

FIG. 14 shows pseudo-code/work flow of draft reads selection by hierarchical rankings and choosing the best overall scoring draft read as the final read, in accordance with the present disclosure;

FIG. 15 shows pseudo-code/work flow of the local best score algorithm, in accordance with the present disclosure;

FIG. 16 shows strategy for De novo sequencing of Fragment III by 2-D LC/MS, in accordance with the present disclosure;

FIG. 17 shows strategy for De novo sequencing of Fragment I by 2-D LC/MS, in accordance with the present disclosure;

FIG. 18 shows strategy for De novo sequencing of Fragment II by 2-D LC/MS, in accordance with the present disclosure;

FIG. 19 shows comparison between final sequences reading out from the same data of Fragment I of tRNA by applying both Global Hierarchical Ranking Strategy and Local Ranking Strategy, in accordance with the present disclosure;

FIG. 20 is a flowchart of a method for determining an order of nucleotides of an RNA molecule in accordance with the disclosure; and

FIG. 21 shows sequence fragment/section assembly by overlapping regions for a complete sequence.

Further details and aspects of exemplary embodiments of the disclosure are described in more detail below with reference to the appended figures. Any of the above aspects and embodiments of the disclosure may be combined without departing from the scope of the disclosure.

### DETAILED DESCRIPTION

Although the present disclosure will be described in terms of specific embodiments, it will be readily apparent to those skilled in this art that various modifications, rearrangements, and substitutions may be made without departing from the spirit of the present disclosure. The scope of the present disclosure is defined by the claims appended hereto.

For purposes of promoting an understanding of the principles of the present disclosure, reference will now be made to exemplary embodiments illustrated in the drawings, and specific language will be used to describe the same. It will nevertheless be understood that no limitation of the scope of the present disclosure is thereby intended. Any alterations and further modifications of the inventive features illustrated herein, and any additional applications of the principles of the present disclosure as illustrated herein, which would occur to one skilled in the relevant art and having possession of this disclosure, are to be considered within the scope of the present disclosure.

For automation of RNA sequencing, algorithms with improved accuracy are needed. The present disclosure relates to development of an algorithm for use with mass RNA laddering sequencing methods (for example, those described in U.S. Patent Ser. No. 62/833,964 which is incorporated herein by reference in its entirety). For a detailed discussion of LC/MS-based RNA sequencing, reference may be made to U.S. Patent Ser. No. 62/833,964 and “A general LC/MS-based RNA sequencing method for direct analysis of multiple-base modifications in RNA mixtures,” Zhang et. al. (available at https://doi.org/10.1101/643387), the entire contents of which are incorporated by reference herein.

RNA sequencing is the process of determining the nucleic acid sequence—the order of nucleotides in RNA. It includes any method or technology that is used to determine the order of the four bases: adenine, guanine, cytosine, and uracil. In addition to the determining the nucleic acid sequence, the methods disclosed herein can also identify, locate, and quantify RNA modifications within the nucleic acid sequence.

The disclosed algorithm includes computational simulations resulting in reciprocal verification between experimental data and simulated data. The simulation provides a means for probing RNA molecules of increased length as well as diverse RNA samples having a mixture of RNA. A hierarchical clustering algorithm has been used to automate RNA sequence generation from the monoisotopic mass data obtained for example, from Agilent's molecular feature algorithm. Although an example Python-based algorithm works well on short RNAs, it was found that when running LC/MS data from tRNA, it slowed down significantly and the error rates increased in the algorithm-generated RNA sequences, likely due to the increased computational workload from the datasets and complexity of the tRNA samples. The 76 nucleotide long tRNA is substantially longer than 20 nt RNAs for which this algorithm was originally derived. Furthermore, the tRNA has 11 different chemical modifications (see Table 1 below). The increase in both chemical modifications and RNA length not only challenged capacity of the Python-based algorithms, but also make the error rate issues pronounced. For short RNA with ˜20 nucleotides long, one can manually calculate the mass differences between two adjacent ladder components to verify accuracy of each sequence readout from the algorithm. For longer RNA, this manual verification becomes more challenging and less efficient. For automation of RNA sequence generation and modification analysis, the development of more robust methods will provide a means for verifying the accuracy of MS-based sequencing data, especially as sequencing of more complicated and longer cellular RNA samples progresses. The algorithm disclosed herein is designed to improve the accuracy of RNA sequencing methods via a two-way sequencing reconfirmation for better accuracy. The algorithm comprises the steps of (i) reading out from MS data to proposed draft sequence reads, (ii) simulation from the proposed draft sequence reads into ideal ladder patterns, and (iii) re-affirmation to see how well they fit.

TABLE 1
Summary of modified bases identified through sequencing of tRNA by LC/MS.

| No. | Modifications | Symbol | Composition | Cal Mass | Detection Method |
|---|---|---|---|---|---|
| 1 | m²G | 2 | C11H15N5O5 | 359.0631 | Not eligible for aniline |
| | | | | | cleavage |
| 2 | m⁷G | 7 | C11H15N5O5 | 359.0631 | Eligible for aniline cleavage |
| 3 | Ψ | P | C9H12N2O6 | 306.2553 | CMC conversion to |
| | | | | | distinguish from U |
| 4 | Cₘ | Cₘ | C10H15N3O5 | 319.0569 | Not eligible for acid |
| | | | | | degradation |
| 5 | Gₘ | Gₘ | C11H15N5O5 | 359.0631 | Not eligible for acid |
| | | | | | degradation |
| 6 | Y | Y | C21H28N6O9 | 570.1475 | Conversion to Y′ under |
| | Y′ | Y′ | C5H9O7P | 212.0086 | acid degradation |
| 7 | D | D | C9H14N2O6 | 308.0410 | By unique mass |
| 8 | m²₂G | J | C12H17N5O5 | 373.0787 | By unique mass |
| 9 | m⁵C | Z | C10H15N3O5 | 319.0569 | By unique mass |
| 10 | T | T | C10H14N2O6 | 320.0410 | By unique mass |
| 11 | m¹A | K | C11H15N5O4 | 343.2358 | By unique mass |

Although, MS-based RNA sequencing methods control degradation conditions to generate well-defined mass ladders for sequencing, the process of generating ladder fragments in the chemical/enzymatic degradation step can lead to the creation of internal fragments that do not possess a 3′ or 5′ end. Use of the algorithm disclosed herein provides a means for utilizing the internal fragments for sequence alignment by piecing them together via clustering undesired RNA oligonucleotide fragments and computational simulation. The algorithm of the disclosure, also helps to increase the accuracy of sequence alignment for RNA with long sequences when fragmentation is utilized to produce shorter RNAs for use in, for example, MS-based sequencing.

In one aspect, the algorithm of the disclosure may be used in conjunction with a variety of different RNA sequencing methods. One such non-limiting method comprises the steps of: (i) affinity labeling of the 5′ and 3′ end of the RNA molecules; (ii) random degradation of the labeled RNA; (iii) optionally, 5′ and 3′ end labeled fragment separation; (iv) separation of resultant target RNA fragments using reverse-phase high performance liquid chromatography (HPLC); and (iv) sequential analysis of resultant mass ladders with high-resolution mass spectrometry for sequence/modification identification. Such an RNA sequencing method is based on the formation and sequential physical separation of two ladder pools of degraded RNA fragments, referred to herein as 5′ and 3′ ladder pools, which are then subjected to LC/MS for HPLC and MS determination of the RNA sequence as well as the presence, type, location and quantity of RNA modifications. The algorithm disclosed herein, is advantageously utilized to analyze the obtained LC/MS derived data.

In one aspect, the algorithm of the present disclosure may be used in conjunction with a variety of different RNA sequencing methods. One such non-limiting method comprises the steps of: (i) chemical labeling of the 5′ and 3′ end of the RNA molecules with different tags; (ii) random degradation of the labeled RNA; (iii) separation of resultant target RNA fragments using reverse-phase high performance liquid chromatography (HPLC); and (iv) sequential analysis of resultant mass ladders with high-resolution mass spectrometry for sequence/modification identification.

The disclosed algorithm recognizes the identities and locations of not only the four canonical ribonucleotides, but also different types of modified ribonucleotides, by their own and/or in their sequential orders, based on the fact that all types of nucleotides have their unique mass and retention time (RT) features in LC-MS data. The algorithms automatically generate sequences that reveal the presence, type, location and quantity of a wide spectrum of different RNA modifications. The algorithms take advantage of the LC/MS characteristic features, including mass and retention time (RT), volume, and quality score for generate sequence reads, and are able to de novo generate the RNA sequences revealing the identity and location of each canonical ribonucleotides and non-canonical base modifications. The data used for the algorithm development including the mass, RT, volume and quality score (QS) were directly exported from LC/MS workstation without any other processing. The algorithms were tested on tRNA (tRNA (phenylalanine specific from brewer's yeast), and their sequence readouts was verified to be accurate.

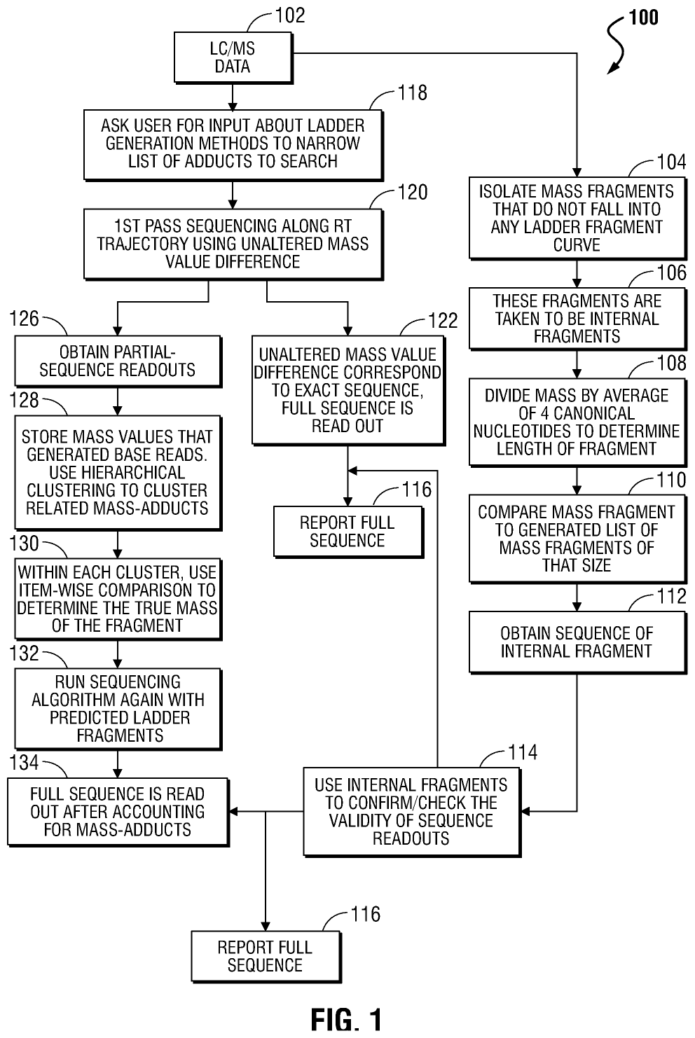

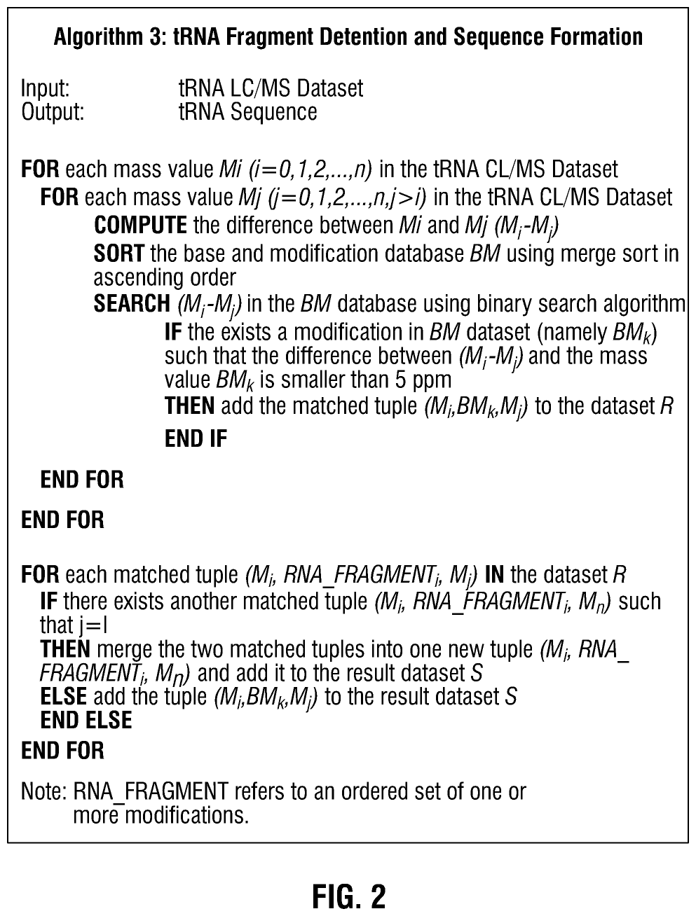

With reference to FIG. 1, a flowchart for the sequencing workflow of the algorithm is shown, in accordance with the present disclosure. In the algorithm disclosed herein (FIG. 1), several steps are taken to use the strengths of the LC/MS data 102 advantageously and to account for the amount of “noise” that may be present in the data. In a first step 104, the data is filtered based on mass, eliminating masses that are too small to be useful in sequencing. Then, at step 106 the remaining data points are sequenced based on mass differences between adjacent ladder fragment compounds that are close together in RT. Starting at a random compound, the algorithm identifies a neighboring compound that is close in RT and calculates the mass difference between the two (see FIG. 2). As used herein, the term RNA fragment or ladder fragment is one compound that was measured by LC/MS; that is also one dot in a 2-D mass-RT plot. At step 108, if the mass difference matches the mass of one of the four canonical nucleotides: A, U, C, G, or a modified base from a database of over 110 known modified RNA bases, the base is stored as a part of sequencing read. The algorithm then continues following the same set of rules for finding the next compound, until no more valid compounds can be found or no more compounds can be found that will produce a mass difference that yields a valid canonical nucleotide or modified nucleotide. If the algorithm is able to read out all of the base-pairs 122, then the sequence is reported 116. In preferred embodiments a natural full length RNA sequence is determined. If there are any gaps in the sequence, then the algorithm proceeds to an auxiliary step.

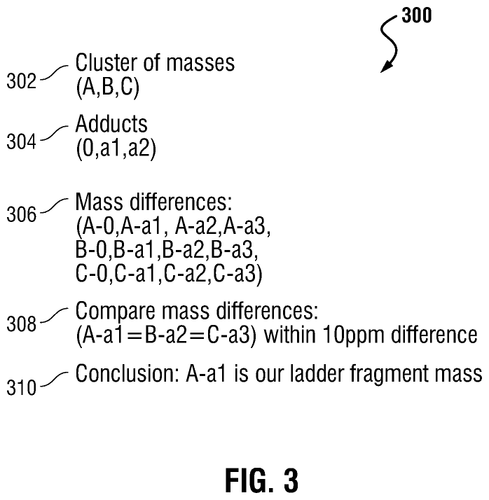

In the auxiliary step, a hierarchical clustering algorithm 128 is used to identify related mass-adducts. In various embodiments, using a distance metric that factors into account the mass as well as RT, the hierarchical clustering algorithm 128 groups compounds based on their mass-relationship so that each cluster contains possible mass-adducts of a true ladder fragment. To cut down on the complexity of the data, points that have already been sequenced in the previous step, and thus subsequently their related mass clusters, will be excluded from the hierarchical clustering step. At step 130, once mass clusters have been identified, the masses will be tested against the masses of the adducts to determine the true mass of the ladder fragment that gave rise to the different mass-adduct fragments. The algorithm will create a new data point with the mass equal to the mass of the ladder fragment identified through the formula in FIG. 3 and RT equal to the average of the RTs in that mass cluster. After new masses are identified through the clustering step, the sequencing algorithm is run again 132 to generate new sequencing reads. Finally, the sequencing reads from the two steps are combined to generate a complete readout of the sequence 134.

With reference to FIG. 3, a formula to determine the mass of ladder fragments obscured by mass-adducts is shown, in accordance with the present disclosure. Initially at step 302, a cluster of masses is determined. For example the cluster of masses may comprise masses A, B, and C. Next at step 304 adducts are determined. For example, 0, a1, and a2. Next at step 306, mass differences are determined. Next at step 308, the mass differences are compared. For example, A−a1=B−a2=C−a3 are within an approximately 10 ppm difference. At step 310 the mass os equal to the mass of the ladder fragment identified through step 308. For example, A−a1 is the ladder fragment mass.

In the event that there are RNA modifications on the 2′-hydroxyl group that block acidic degradation, a different approach will be adopted to fill the gap caused by the blocking group at the 2′-O position. RNA modifications, e.g., methylation on the 2′-hydroxyl group of RNA, render the adjacent 3′-5′-phosphodiester linkage non-hydrolysable, create a mass gap in both the 5′- and the 3′-mass ladder families that are larger than one nucleotide. As a result, it is determined that there is a single modification on the 2′-O position and the combination of two nucleotides, but their order is unknown. To resolve such ambiguities, the computational simulation is used to match the observed LC/MS data 102 against the simulated 2′-O-modified sequence, and thus the results from these analyses should match well if there is a modification at 2′-O-position. In addition, the complete nucleotide sequence can be assembled through conventional RNA sequencing platforms. Alternatively, collision induced dissociation (CID) MS can be performed on the 2′-O-modified dimer fragment to elucidate the structure of the dinucleotide fragment.

In various embodiments, the last step of the sequencing process is to harness the presence of multiple internal fragments in the data to function as a new sequence or a check for the final sequence. Masses that are not included in the mass clusters or used in the sequencing reads are divided by the average value of the four canonical bases to estimate their sequence length. In various embodiments, sequences from 3 to 6 bases in length are compared to a list of generated masses of internal fragments that are 3 to 6 bases in length to find a precise match t. These short fragments can be used to fill gaps in the sequence or confirm the accuracy of the sequence.

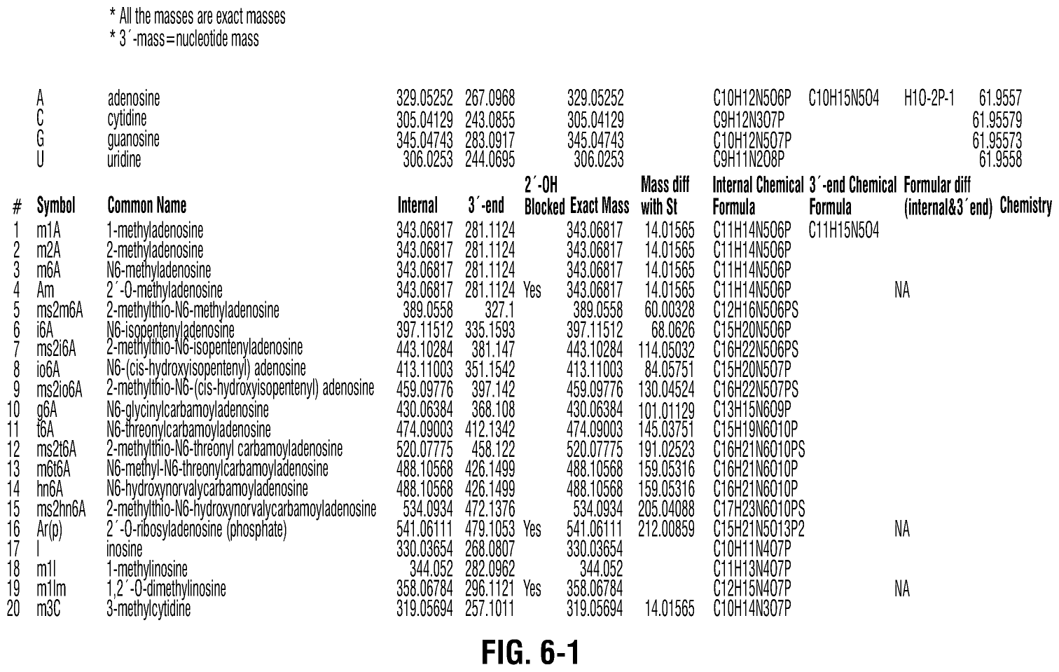

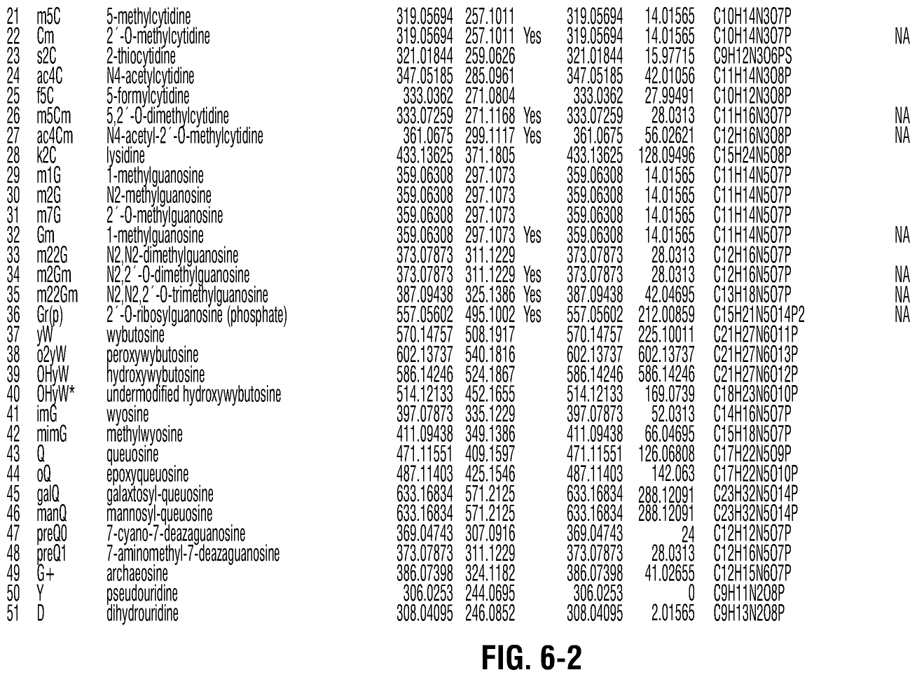

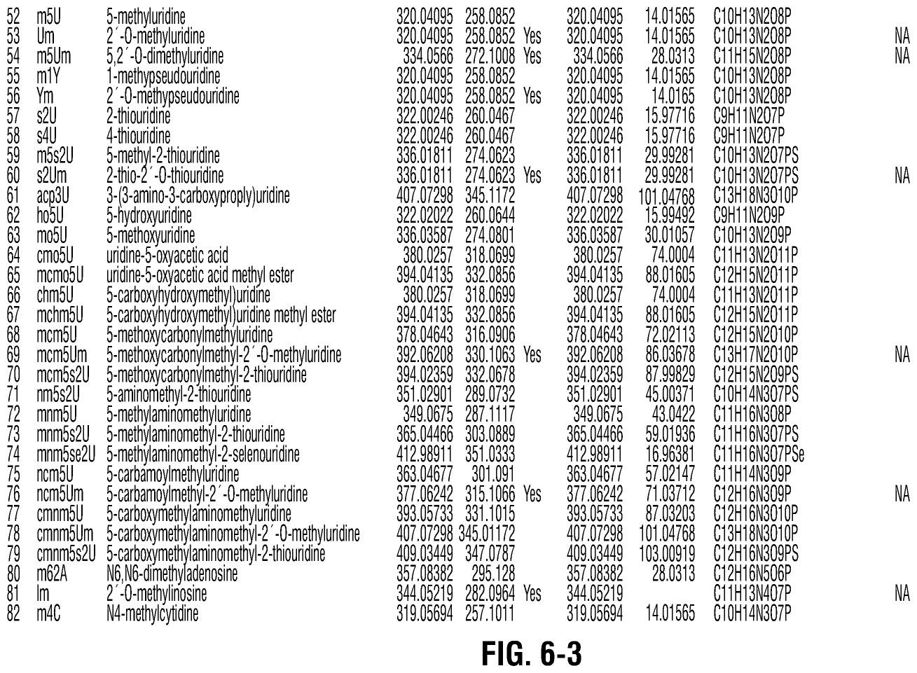

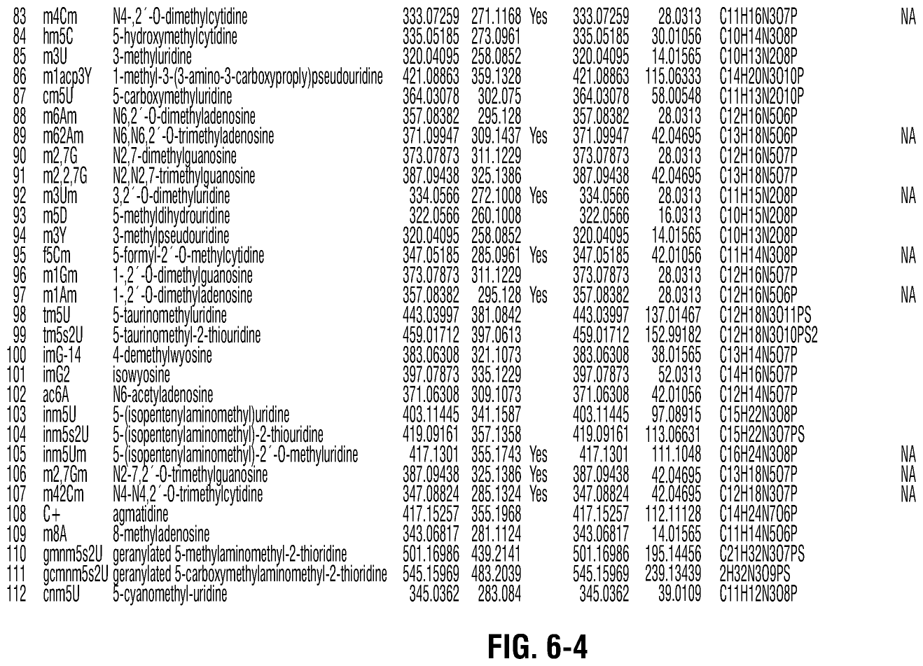

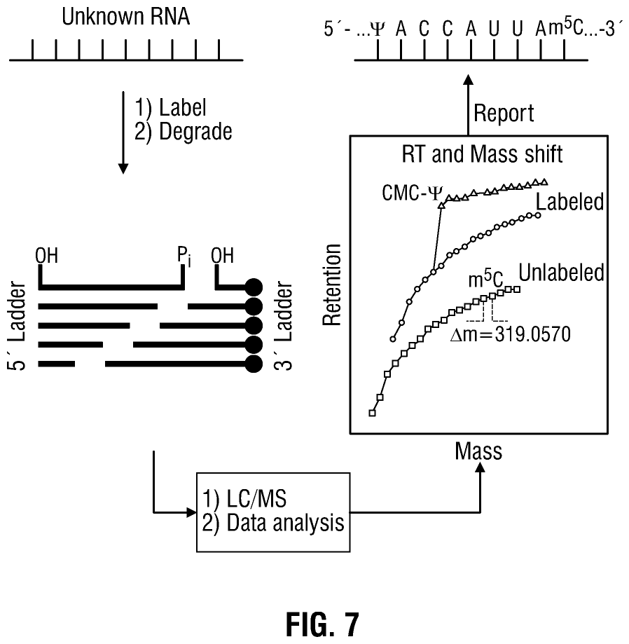

In various embodiments, the raw data derived from LC-MS, which contains the m/z data of the desired fragments and/or the undesired fragments bearing more than one cleavage may be decovoluted over the entire LC run using Agilent's molecular feature algorithm built into MassHunter™ software, which is subsequently used for sequence alignment. Mass adducts can be removed from the deconvoluted data and the sequences will be predicted/generated using both mass and retention time data. The retention time-coupled m/z data for the fragments is analyzed and classified using a developed support vector machine (SVM) classifier algorithm; to determine which data points are “valid” and to be used for subsequent sequence determination and which data points are to be filtered out. After data reduction step, the mass difference (m) between two adjacent RNA ladder fragments [m=m (i)−m(i−1), 1<i<n, n=RNA length], where m(i) is the mass of any ladder fragment and m(i−1) is the preceding lower mass ladder fragment, and match such mass differences with the exact masses of known nucleotide fragments using search algorithms designed-to correlate the derived RNA sequencing information based on mass differences to determine the identity of canonical nucleotides and their modification. As long as the structural modification on an RNA nucleoside is mass-altering, the search algorithms and the dynamic programming method together will permit identification of the RNA sequence and its modification to be identified. In various embodiments, the mass of the known modified ribonucleotides can be conveniently retrieved from known RNA modification database or through use of the table shown in FIG. 6.

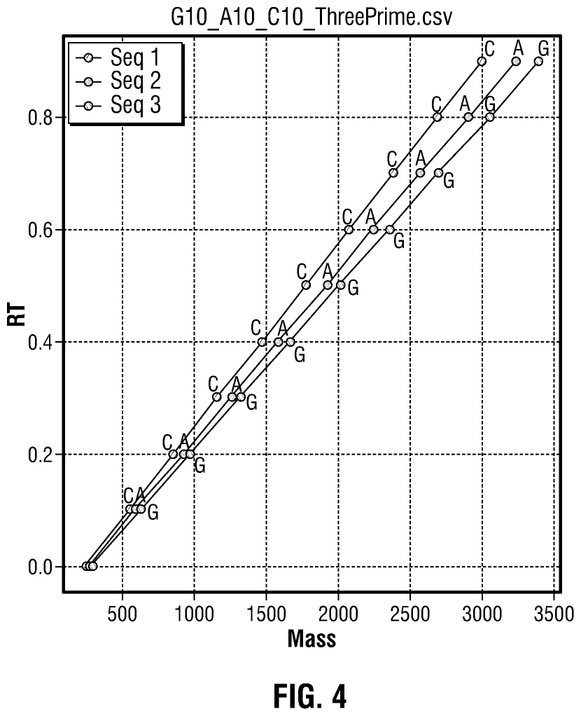

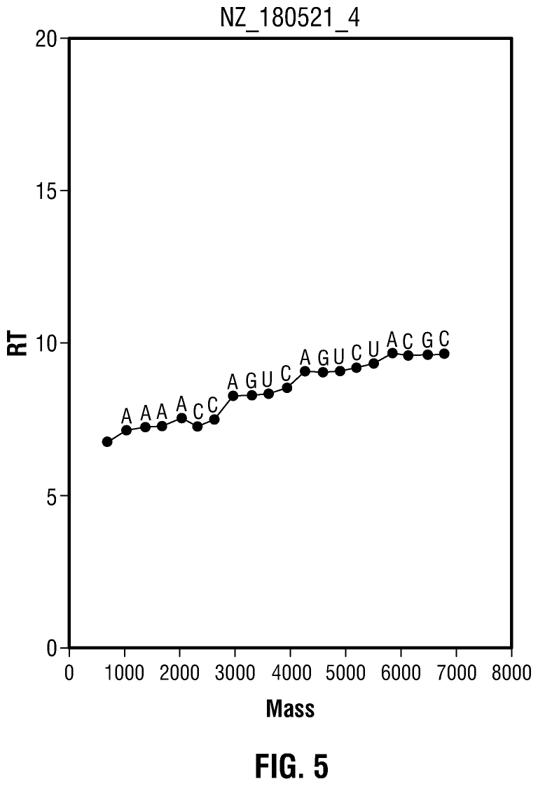

With reference to FIG. 4, computational simulation of the simultaneous base-calling of 3′-mass ladder fragments of three homopolymers is shown, in accordance with the present disclosure. In addition to utilization of the undesired fragments with more than one cut for sequence alignment, a simulation is introduced to train the algorithms for automation of RNA sequence generation to increase the sequencing accuracy. An MS library of RNA with random sequences, both in the laboratory and in silico was constructed, and the algorithms tested on sequence generation. The difficulty was increased stepwise by bringing in, e.g., chemical modifications and multiple RNA strands (FIG. 4). In addition, the algorithms were tested on read length and throughput both in the laboratory and in silico to enable sequencing mixed RNA samples and sequence readouts were compared from theoretical/simulation and experimental data.

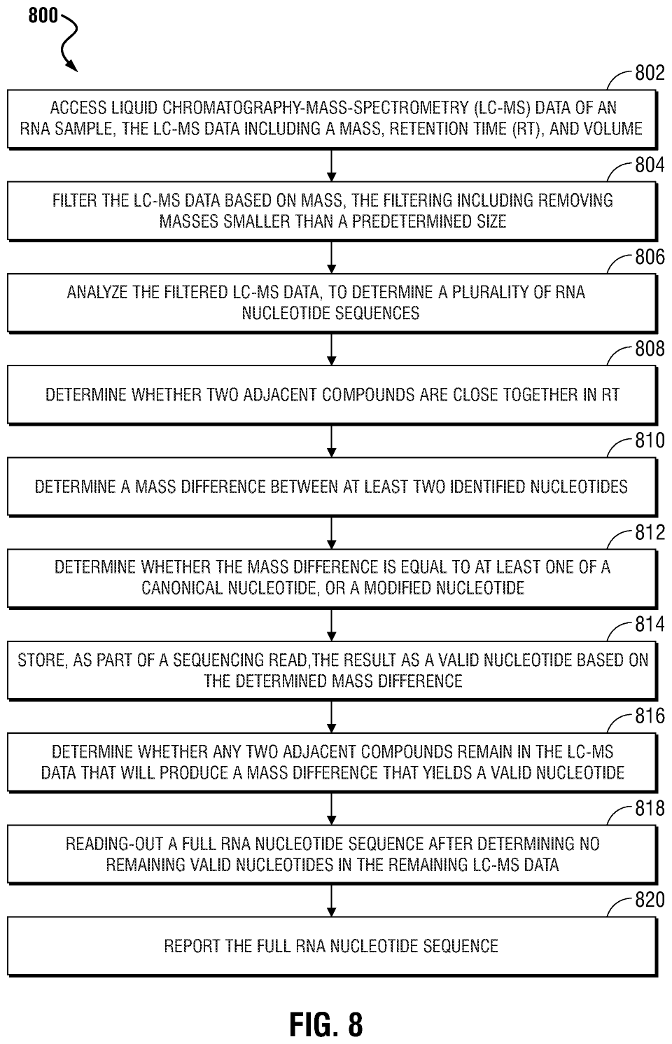

With reference to FIG. 8, a flow diagram is shown, which is illustrative of a method 800 for determining an order of nucleotides of an RNA molecule in accordance with the present disclosure. Initially at step 802 the system receives liquid chromatography-mass-spectrometry (LC-MS) data of an RNA sample. The LC-MS data includes a mass, retention time (RT), and volume. In various embodiments, a length of the RNA molecule is more than 20 nucleotides. In various embodiments, one or more RNA molecules are present in the RNA sample to be sequenced. In various embodiments, the RNA sample may include a purified RNA sample of limited diversity. In various embodiments, the RNA sample may include a therapeutic RNA molecule.

Next, at step 804, the system filters the LC-MS data based on mass, the filtering including removing masses smaller than a predetermined size. In various embodiments, the data is filtered based on mass, eliminating masses that are too small to be useful in sequencing.

Next, at step 806, the system sequences the filtered LC-MS data, to generate an RNA sequence. The sequencing includes steps 808 thru 812. At step 808, the system determines whether two adjacent compounds are close together in RT. Next, at step 810, the system determines a mass difference between the two adjacent ladder fragments. In various embodiments, the system may, starting at a random compound, identify a neighboring compound that is close in RT and calculates the mass difference between the two (See FIG. 2).

Next, at step 812, the system determines whether the mass difference is equal to at least one of a canonical nucleotide, or a modified nucleotide. In various embodiments, the system determines whether the mass difference matches the mass of one of the four canonical nucleotides: A, U, C, G, or a modified base from a database of over 110 known modified RNA bases. Next, at step 814, the system stores in a memory, as part of a sequencing read, the result as a valid nucleotide based on the determined mass difference.

Next, at step 816, the system determines whether any two adjacent compounds remain in the LC-MS data that will produce a mass difference that yields a valid nucleotide. In various embodiments, the algorithm then continues following the same set of rules for steps 808 thru 812 for finding the next compound, until no more valid compounds can be found or no more compounds can be found that will produce a mass difference that yields a valid canonical nucleotide or modified nucleotide. In various embodiments, the system determines if it is able to read out all of the base-pairs. In various embodiments, if there are any gaps in the sequence, then the algorithm proceeds to an auxiliary step.

In various embodiments, in the auxiliary step the system determines whether there are any remaining compounds that did not yield a valid nucleotide based on the gaps. If there are any gaps, the system performs a hierarchical clustering algorithm on the compounds to identify related mass-adducts. In various embodiments, the hierarchical clustering algorithm includes determining a distance metric based on a mass as well as RT for the compound, grouping compounds, into a cluster of masses, based on their mass relationship, such that each fragment includes possible mass-adducts of a true ladder fragment. In various embodiments, points that have already been sequenced in the previous step, and thus subsequently their related mass clusters, will be excluded from the hierarchical clustering step.

In various embodiments, the system then determines the mass of a fragment for each cluster, based on item-wise comparison between the identified mass-adducts and the cluster of masses. In various embodiments, the system then predicts a ladder fragment based on the determined mass for each cluster. In various embodiments, the system then reads-out an RNA sequence based on the predicted ladder fragment, and reports the RNA sequence

Next, at step 818, the system reads-out an RNA sequence based on determining there are no remaining valid nucleotides in the remaining LC-MS data. Next, at step 820, the system reports the RNA sequence. In various embodiments, the system may display on a display the RNA sequence.

In various embodiments, liquid-chromatography-mass spectrometry-(herein referred to as LC-MS) based RNA sequencing method may be used to simultaneously determine the nucleotide sequence of a target RNA molecule with single nucleotide resolution, as well as, detect the presence of target RNA modifications. The disclosed method can be used to determine the type, location and quantity of each modification within the target RNA sample. Such techniques can be used advantageously to correlate the biological functions of any given RNA molecule with its associated modifications and for quality control of RNA-based therapeutics.

In various embodiments, the above method 800 of FIG. 8, may include liquid chromatography-mass-spectrometry (LC-MS) based RNA sequencing techniques that rely on end-labeling of RNA to be sequenced with a hydrophobic tag like biotin either at its terminal 5′ end or at its terminal 3′-end, and on the subsequent generation of fragmented ladder RNA. In various embodiments, the method 800 take advantage of the characteristic LC/MS features, including mass and retention time (RT), volume, and quality score, to de novo generate the RNA sequences revealing the identity and location of each canonical ribonucleotides and non-canonical base modifications. The method 800 may include generating sequences that reveal the presence, type, location and quantity of a wide spectrum of different RNA modifications.

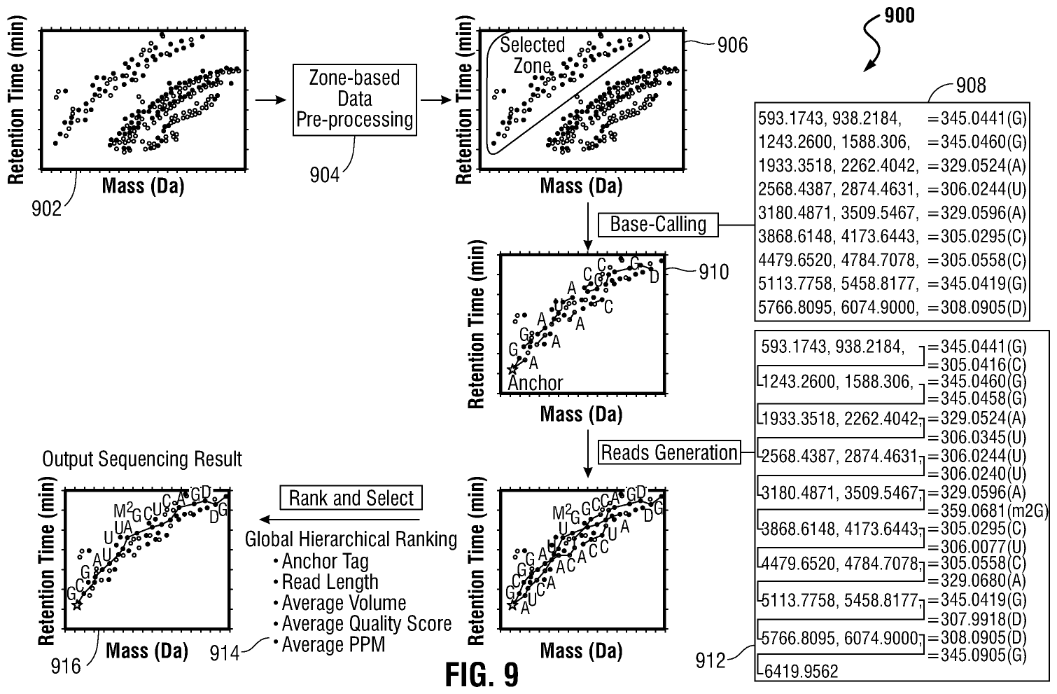

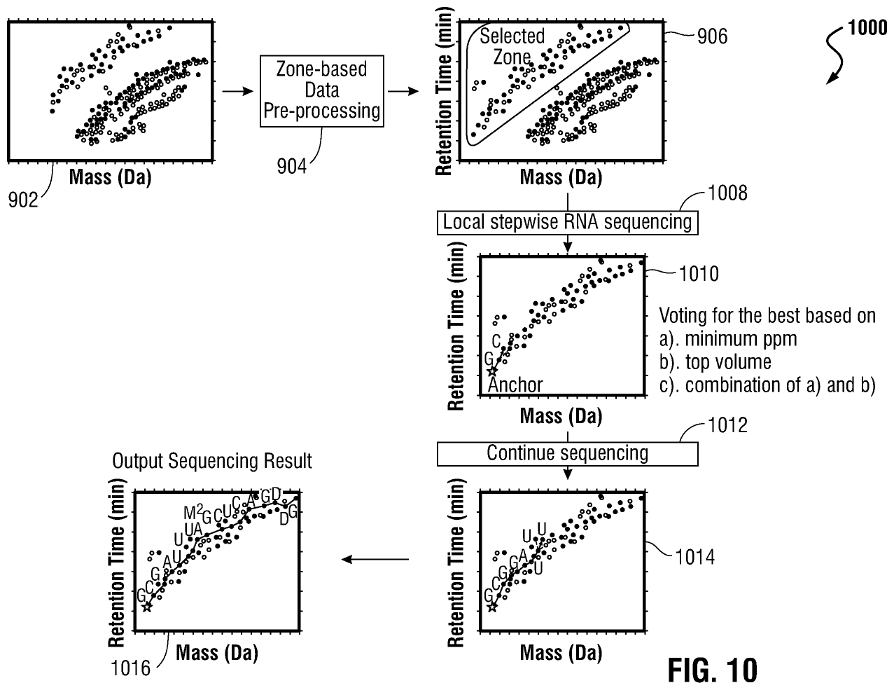

With reference to FIGS. 9 and 10, methods for performing a draft read strategy are shown. In various embodiments, the algorithms perform data pre-processing, base calling, sequence generation and output filtering on the input dataset, which is the output from the LC-MS formatted in a specific manner. For example, the sample data was acquired using the MassHunter™ Acquisition software (Agilent Technologies™, USA). To extract relevant liquid chromatographic and mass spectral (LC-MS) information from the data collected from the LC-MS experiments, the Molecular Feature Extraction (MFE) workflow in MassHunter™ Qualitative Analysis (Agilent Technologies™, USA) was used. This proprietary molecular feature extractor (MFE) algorithm performs untargeted feature finding all the possible compounds each with its unique mass and retention time dimensions. The MFE settings of the software were varied depending on the amount of RNA used in the experiment. The MFE settings we applied were as follows: “centroid data format, small molecules (chromatographic), peak with height ≥500, up to a maximum of 1000, quality score ≥30”. There are two variations of the algorithm implementing the global hierarchical ranking strategy and the local best score strategy respectively (FIG. 9 and FIG. 10). It is contemplated that other software may be used.

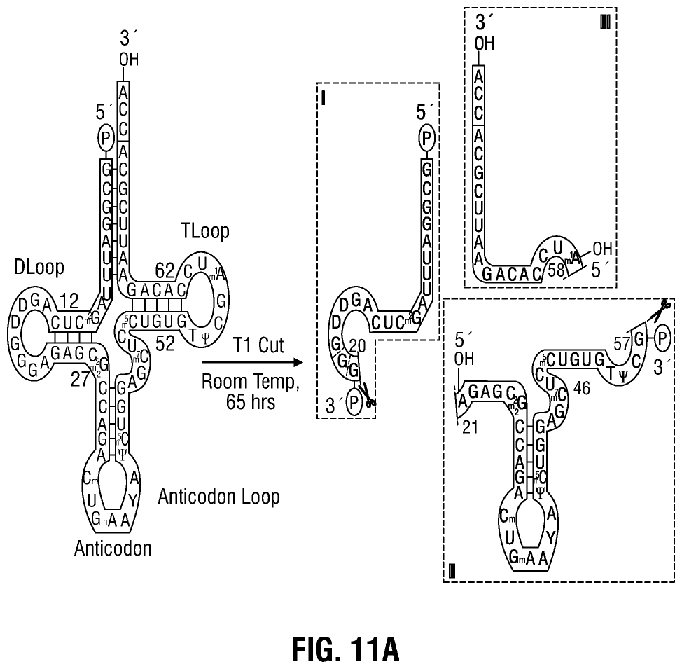

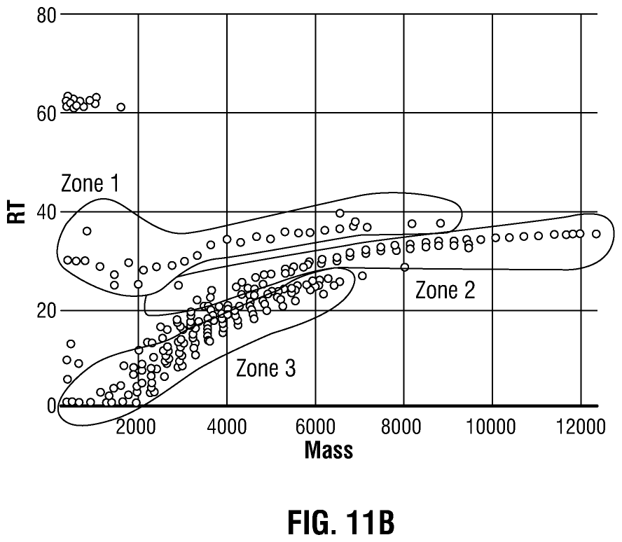

With reference to FIG. 11A, a generation of three major fragments by RNase T1 digestion of tRNA detected by LC/MS, Fragment I, II, and III is shown, in accordance with the present disclosure. With reference to FIG. 11A, a selection of data zones 906 in the 2-D RT versus mass plot of test tRNA sequencing output dataset is shown, in accordance with the present disclosure. Data pre-processing 904 is a step in order for the algorithm to focus on a particular subset of the input dataset at a time by selection a data zone 906, e.g., the top zone in which all the mass ladder components have a biotin tag. The hydrophobicity of the biotin label causes a significant increase in RT values of the ladder components when compared to the unlabeled ladder components.

In various embodiments, there are at least two reasons to subset the dataset 904 before parsing into the algorithm. First is to identify mass ladders needed for sequencing and to eliminate noise data from the dataset. Second is to make the algorithm easy to process a partial dataset, rather than the complete dataset. In various embodiments, it is possible because we have introduced a hydrophobic tag like biotin or Cy3 to the RNA to be sequenced experimentally. The hydrophobicity of the label causes a significant increase in RT values of the ladder components when compared to the unlabeled ladder components, and help all the labeled mass ladder components upshift to the top zone so that we can easily identify labeled mass ladders in the 2-D mass-RT plot. Here we show the graphical distribution of data points from the test tRNA sequencing (FIGS. 11A and 11B). The algorithm “zooms in” on one group to read out the sequence of one fragment at a time. Subsetting of the dataset is implemented by refining the RT and mass value of the input dataset in windows, and specifying the starting data point of each fragment. This is feasible because the molecular tag is added to the terminus of each fragment, and the RT and mass feature of the tag is known. Therefore, the algorithm is called anchor-based, since specifying the starting data point corresponding to the molecular tag latches down the data points corresponding to the fragment from the whole dataset.

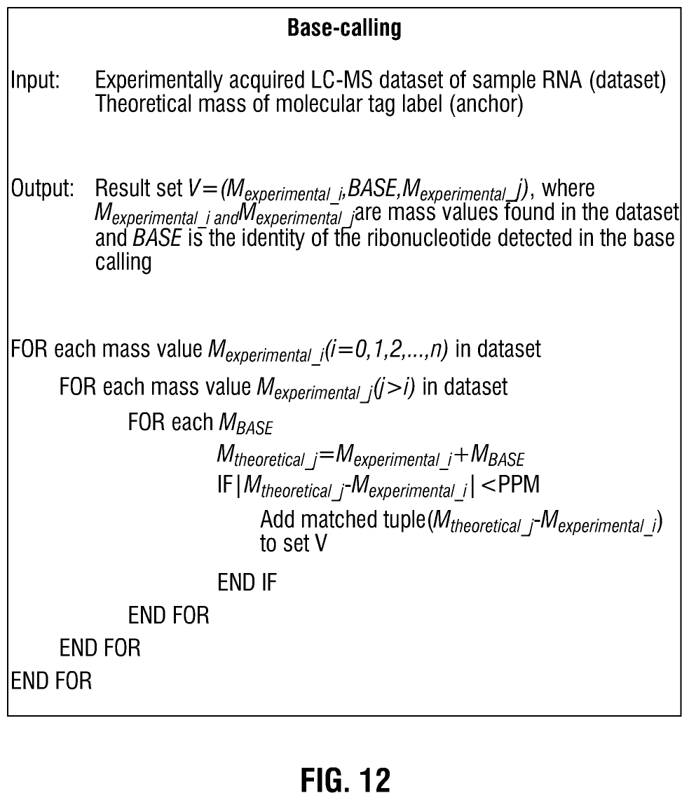

With reference to FIG. 12, pseudo-code of base calling 908 is shown, in accordance with the present disclosure. After subsetting the dataset, the algorithm performs base calling 908. The theoretical mass, calculated from chemical formula, of all known ribonucleotides including those with modifications to the base is stored as a list of M_{BASE}. In the first iteration the algorithm finds the mass corresponding to the molecular tag (anchor) 910 and sets Mₑₓₚₑᵣᵢₘₑₙₜₐₗ equal to this mass. The algorithm tests each M_{BASE} from the list by adding it to Mₑₓₚₑᵣᵢₘₑₙₜₐₗ and generating a theoretical sum mass M_{theoretical_j}. The algorithm searches through the dataset for a mass value that matches with M_{theoretical_j}. If there exists a matching mass value M_{experimental_j}, a tuple (M_{experimental_i}, BASE, M_{experimental_j}) is stored in the result set V. Since the algorithm tests all M_{BASE} in the list and looks for all possible matches, multiple tuples with same M_{experimental_i} but different BASE identity and M_{experimental_j} are stored in set V. When the algorithm decides if there is a match, it takes into consideration the experimental error that the experimental mass may slightly deviate from the theoretical mass for a same ribonucleotide. We implemented a calculated parameter PPM (parts per million) that allows M_{experimental_j} to be matched with M_{theoretical_j} within a customizable range. The formula for PPM is

$$PPM = \frac{M a s {s}_{experimental} - {Mass}_{theoretical}}{M a s {s}_{theoretical}} \times 1 {0}^{6} .$$

The algorithm performs base calling for all data points until all possible tuples are stored in set V. Note that each tuple in set V represents an individual base-calling possibility.

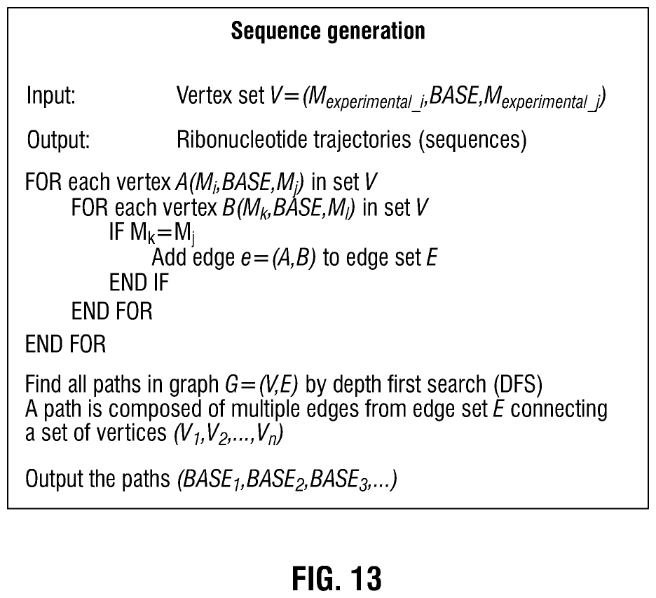

With reference to FIG. 13, pseudo-code/work flow of sequence generation by building trajectories is shown, in accordance with the present disclosure. In various embodiments, after base calling, the algorithm builds trajectories linking tuples in set V to generate sequences of the RNA fragment. Taken tuples from set V as vertices, the algorithm finds and stores all edges by examining pairs of tuples such that for a given pair of tuples (Mᵢ, BASE, Mⱼ) and (Mₖ, BASE, Mₗ), Mₖ=Mⱼ. The algorithm generates a graph G=(V, E) while finding the edges. When graph G is completed, the algorithm finds all paths in graph G by depth first search (DFS). All paths are stored as sets of vertices. Since the vertices contained in the path are tuples (M_{experimental_i}, BASE, M_{experimental_j}), BASE can be outputted as a draft read 912 of RNA sequence.

In various embodiments, because the outputs from LC-MS contains a huge number of data points, graph G contains the same number of vertices and also huge number of edges, resulting in tremendous number of total paths, each representing a draft read. To effectively filter the draft reads for reporting correct sequences, two draft read selection strategies, have been developed namely the global hierarchical ranking strategy 900 and the local best score strategy 1000. Nonetheless, both strategies use same parameters acquired from the LC-MS dataset to score the draft reads 914 which include PPM, RT, volume, quality score (QS), read length.

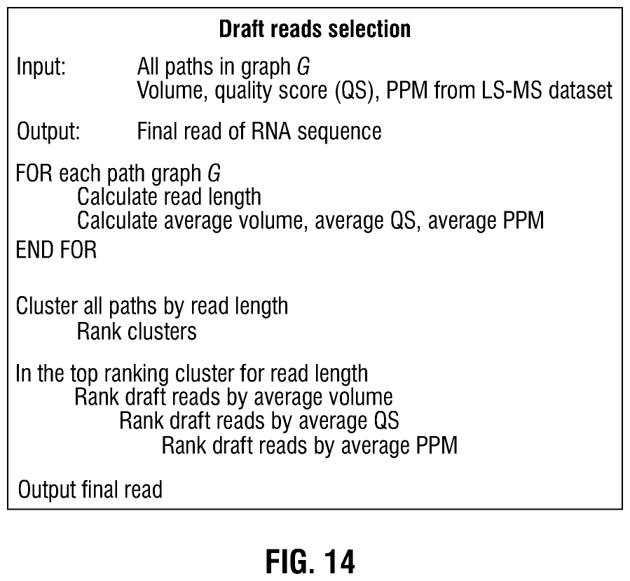

With reference to FIG. 14, pseudo-code/work flow of draft reads selection by the hierarchical ranking strategy 900 and choosing the best overall scoring draft read as the final read is shown, in accordance with the present disclosure. In various embodiments, in the global hierarchical ranking strategy, the draft reads are scored after the sequence generation step with the following criteria: read length, average volume, average QS, and average PPM. Read length is the number of BASE in a draft read. Average volume is calculated by summing the volume associated with each data point in a draft read and diving the sum by read length. Average QS is calculated by dividing the sum of QS by read length for each draft read. Average PPM is the sum of all PPM values associated with data points contained in a draft read divided by read length. The first step of the global hierarchical ranking strategy groups all draft reads into clusters based on their read length, and each cluster is assigned a ranking score for read length. The cluster receiving the highest ranking contains draft reads of the top read lengths, and the algorithm focuses on this cluster in the flowing steps. Within this cluster, the draft reads are assigned secondary ranking scores based on average volume values, with drafts reads of higher average volumes receiving higher rankings. In case where more than one draft read have a same read length and average volume value and thus receive a same ranking, the algorithm uses average QS value to re-rank these draft reads, with higher average QS values resulting in higher ranks. If there are still multiple draft reads receiving the same rank, the algorithm uses average PPM value to re-rank these draft reads again, but higher ranks are assigned to draft reads with lower average PPM values since PPM reflects the difference between the observed mass value and its theoretical mass value associated with each data point of mass ladder components from LC-MS. In the end, the draft read with longest read length, highest average volume, highest average QS and lowest average PPM beats all other draft reads in the hierarchical ranking procedure and will be outputted as the final read of the sequence.

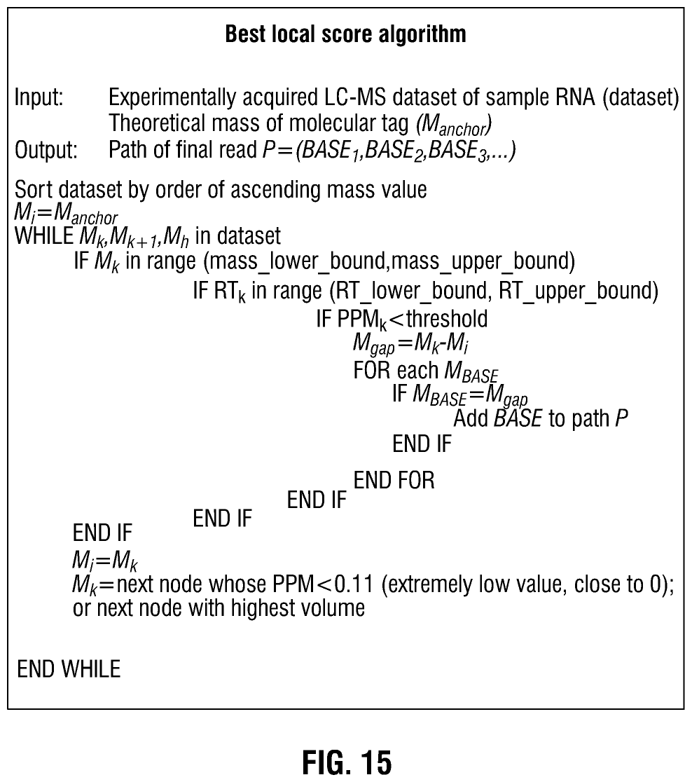

With reference to FIG. 15, pseudo-code/work flow of the local best score strategy 1000 is shown, in accordance with the present disclosure. Alternatively, the local best score strategy 1000 differs from the previous strategy from the step of base calling. In various embodiments, the algorithm of local best score strategy 1000 applies the anchor-based method 1010 to focus on a specific subset of LC-MS dataset presorted by ascending mass order. In various embodiments, it pins down the starting ribonucleotide by user defined anchor mass and locates data points from the entire fragment by the anchor. In various embodiments, focusing on these data points, the algorithm now performs base calling and simultaneously evaluates each data point. In various embodiments, all data points in the desired zone are now considered as nodes, and the algorithm completes a single path as the final read based on the evaluation of each node. For a current node, it's mass difference from the previously node (initialized as the anchor) is compared to the list of all known ribonucleotide masses for a match of identity. The match is only accepted if the PPM value of this node is below a certain threshold. In the test data with tRNA samples, this threshold was specified as 10, but it should always be customized to the actual LC-MS dataset. After accepting or rejecting the match (or mismatch otherwise), the algorithm stores the identity of the matched ribonucleotide, and moves on to the next node. There are always several possible next nodes based on their RT. The node with the highest volume will be chosen, with the exception that if a node has outstandingly small PPM value (close to 0) then this node will be chosen over other nodes with higher volumes. The algorithm now searches for a match of identity of the chosen node, evaluates the match, and store the ribonucleotide identity. This process is repeated until the sequence in the desired data zone is read out. One example of de novo MS sequencing of tRNA^{Phe} from yeast.

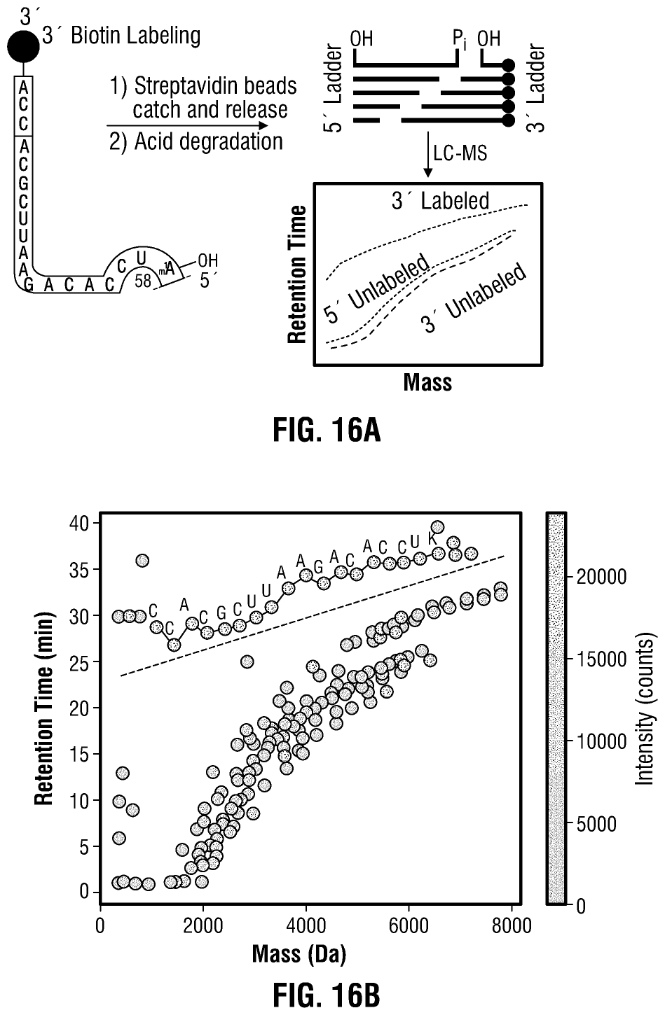

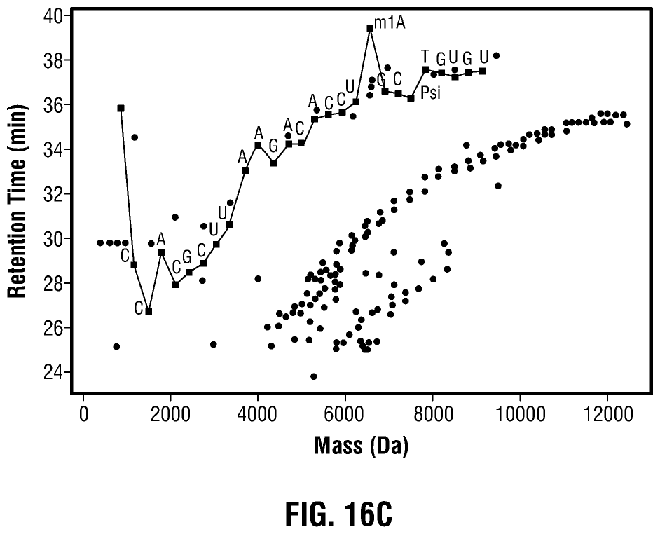

FIG. 16 shows strategy for De novo sequencing of fragment III by 2-D LC/MS. a) 3′ end of fragment III was labeled with a biotin tag by use of A(5′)pp(5′)Cp-TEG-biotin-3′ and T4 RNA ligase. After catch and release with the aid of streptavidin-coupled beads, the resultant fragment III was subjected for acid degradation and subsequent LC/MS analysis. A schematic picture shows/predicts the potential t_{R}-mass shift caused by the biotin tag that was introduced to the 3′ end of all of the ladder components. b) Identifying 3′-biotin-labeled mass ladders of fragment III from 2-D LC/MS data 102 for sequencing. The sequence in the top curve (above the dotted red line) was de novo generated automatically by a Python-coded algorithm using local best score strategy (SI). K: m¹A.

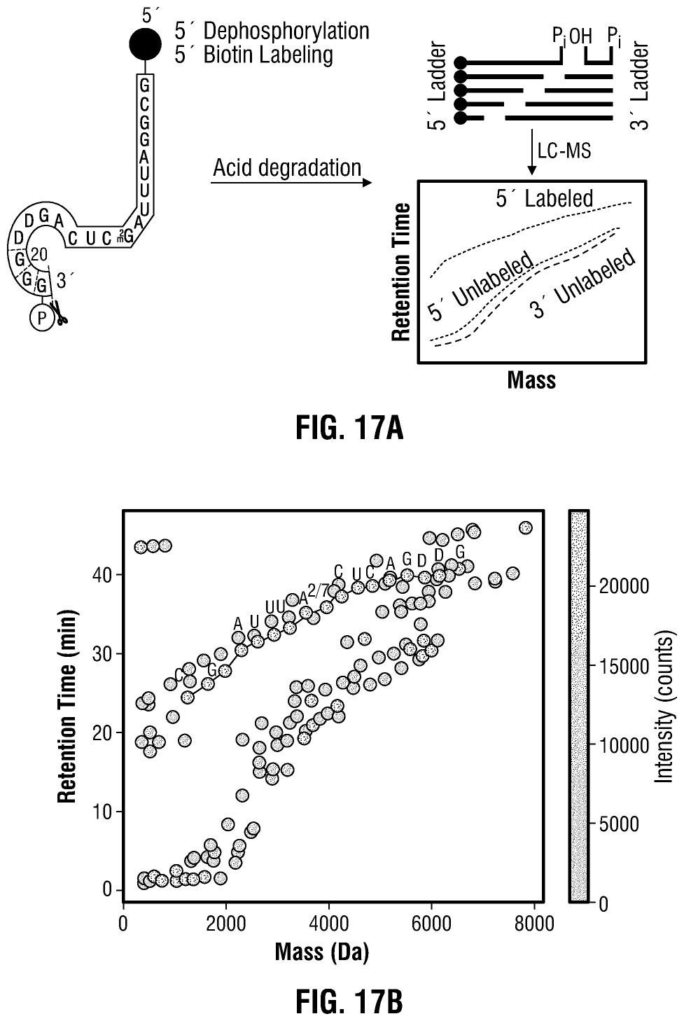

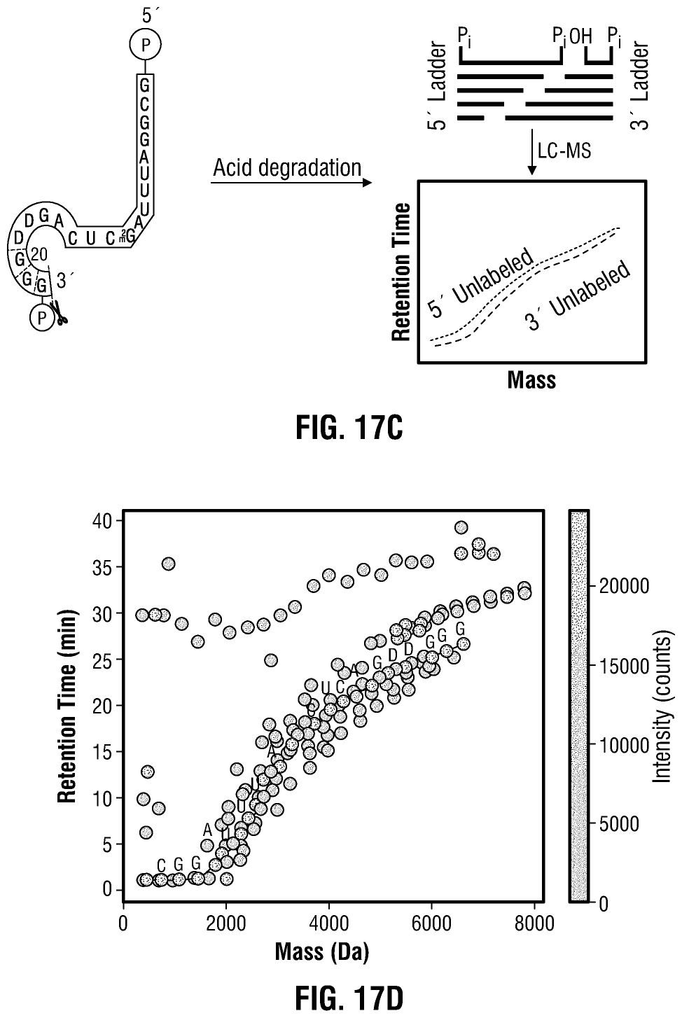

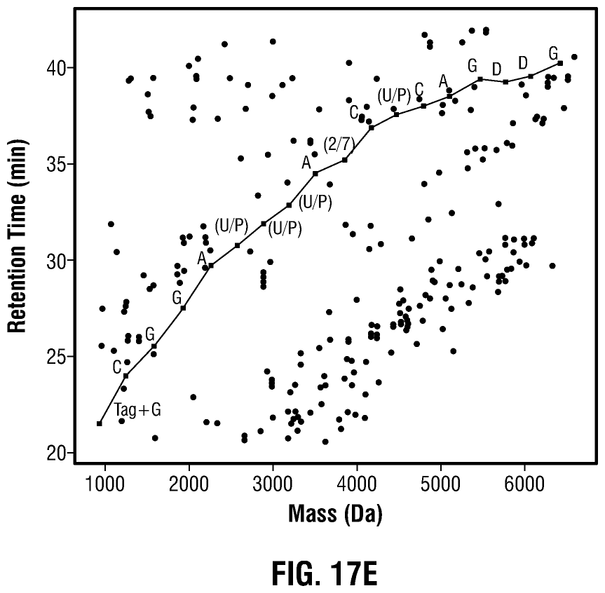

FIG. 17 shows strategy for De novo sequencing of Fragment I by 2-D LC/MS. a) 5′ end of fragment I was dephosphorylated and subsequently labeled with a biotin tag. After catch and release with the aid of streptavidin-coupled beads, the resultant fragment I was subjected for acid degradation and subsequent LC/MS analysis. A schematic picture shows/predicts the potential mass-RT-shift caused by the biotin tag that was introduced to the 5′end of all of the ladder components. b/e) Identifying 5′-biotin-labeled mass ladders of Fragment I from 2-D LC/MS data (above the top red-dotted line) for sequencing. The sequence in the top curve was de novo generated automatically either by a Python-coded algorithm using local best score strategy (b) or JAVA-coded algorithm using the global hierarchical ranking strategy (e). c) Fragment I was directly acid-degraded for LC/MS analysis without any labeling, however, it carries a terminal PO₄⁻ at its 5′end, which can be programmed as a mass tag for de novo generation of the sequence of Fragment I automatically using the Python-coded algorithm using local best score strategy (d).

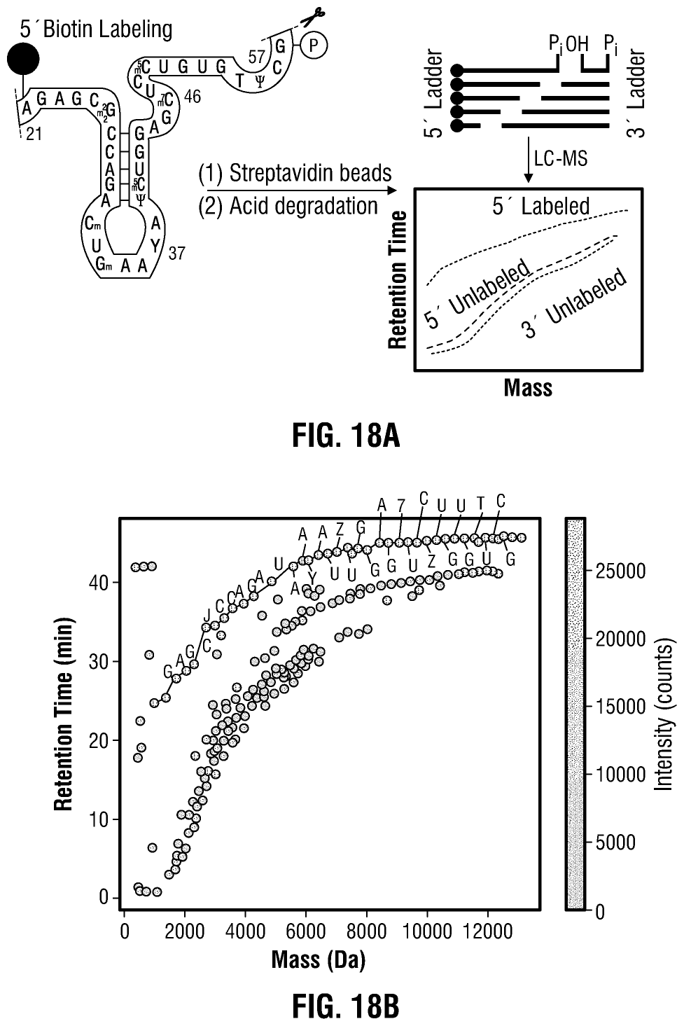

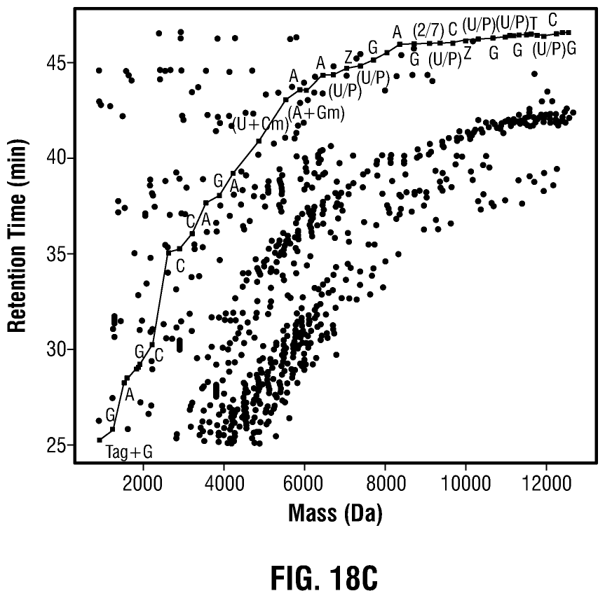

FIG. 18 shows strategy for De novo sequencing of Fragment II by 2-D LC/MS. a) 5′ end of Fragment II was labeled with a biotin tag with a chemistry descripted in the method section. After catch and release with the aid of streptavidin-coupled beads, the resultant Fragment II was subjected for acid degradation and subsequent LC/MS analysis. A schematic picture shows/predicts the potential t_{R}-mass shift caused by the biotin tag that was introduced to the 5′end of all of the ladder components. b-c) Identifying 5′-biotin-labeled mass ladders of Fragment II from 2-D LC/MS data for sequencing. The sequence in the top curve was de novo generated automatically by a Python-coded algorithm using local best score strategy (b) and a JAVA-coded algorithm using the global hierarchical ranking strategy (c).

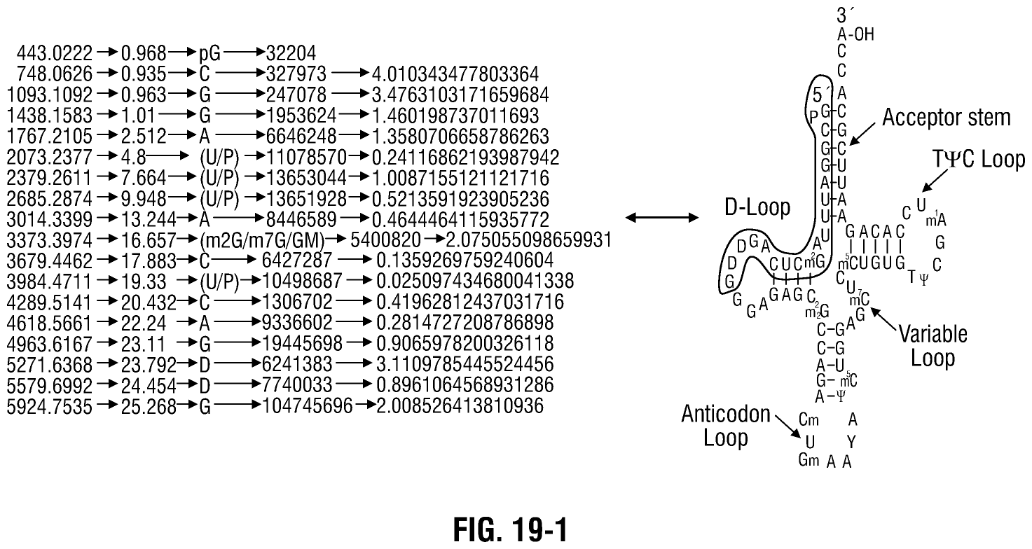

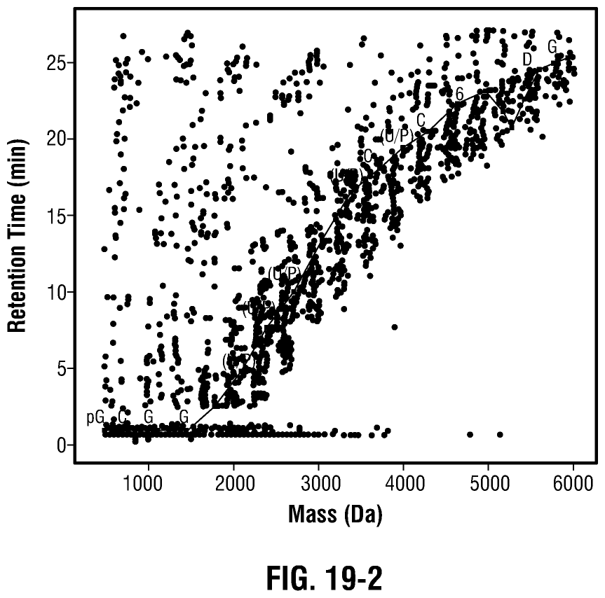

FIG. 19 shows comparison between final sequences reading out from the same data of Fragment I of tRNA by applying both Global Hierarchical Ranking Strategy and Local Ranking Strategy. a) The final sequence read matches perfectly the sequence of the tRNA's Fragment I from the 5′-end, which means that both the global hierarchical ranking can effectively generate sequences. b) A JAVA-coded algorithm using the global hierarchical ranking was applied for de novo generation of the sequence of Fragment I automatically.

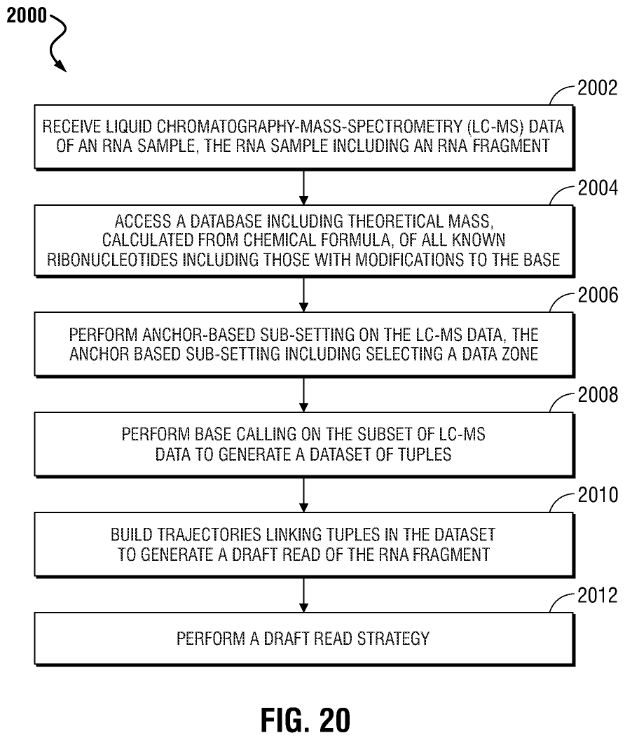

With reference to FIG. 20, a flow diagram is shown, which is illustrative of a method 2000 for determining an order of nucleotides of an RNA molecule in accordance with the present disclosure. Initially at step 2002 the system receives liquid chromatography-mass-spectrometry (LC-MS) data of an RNA sample. The LC-MS data includes a mass, retention time (RT), and volume. The RNA sample includes an RNA fragment. In various embodiments, the computer implemented method further includes biochemical labeling of the RNA sample.

Next at step 2004, the system accesses a database which includes theoretical mass, calculated from chemical formula, of all known ribonucleotides including those with modifications to the base. Next at step 2004, the system performs anchor-based sub-setting on the LC-MS data, the anchor based sub-setting including selecting a data zone.

Next at step 2006, the system performs base calling on the subset of LC-MS data to generate a dataset of tuples. Next at step 2008, the system builds trajectories linking tuples in the dataset to generate a draft read of the RNA fragment. In various embodiments, the draft read strategy includes a global hierarchy ranking strategy or a local best strategy. In various embodiments, the draft read strategy includes a local best strategy. In various embodiments, building trajectories further includes performing a Depth First Search (DFS) algorithm to ensure that all possible draft reads will be found from the LC-MS data.

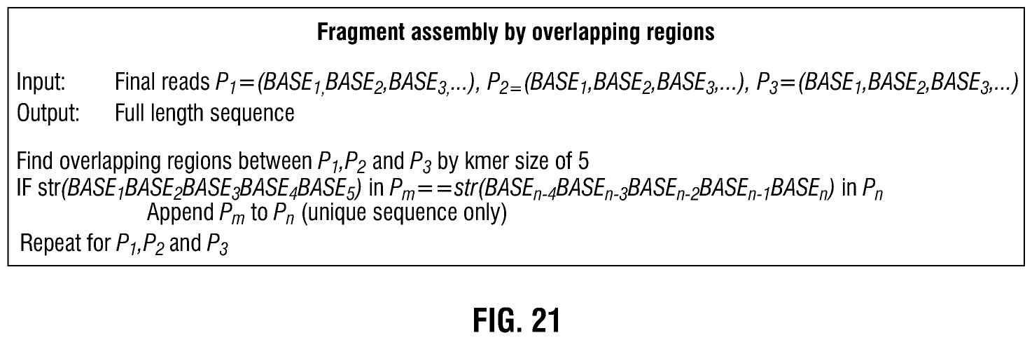

Next at step 2010, the system performs a draft read strategy. With reference to FIG. 21, after performing a chosen draft read strategy, the sequence of the tRNA is assembled based on the overlapping regions of the fragments. If the leading sequence of one fragment aligns with the ending sequence of another fragment at a kmer size of 5, these two fragments are assembled. The kmer size of 5 is chosen based on observation of experimental data that the sequencing reads of fragments of the test tRNA sample contain overlaps of at least 5 bp long, which is a result of designed incomplete fragmentation from sample preparation. The kmer size of 5 is sufficient to guarantee the accuracy of fragment assembly considering the small size of the fragments. The kmer size is also adjustable for different applications other than sequencing tRNAs.

In various embodiments, the draft read strategy includes scoring based on at least one of read length, average volume, average QS, or average PPM.

The systems described herein may also utilize one or more controllers to receive various information and transform the received information to generate an output. The controller may include any type of computing device, computational circuit, or any type of processor or processing circuit capable of executing a series of instructions that are stored in a memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, causes the one or more processors to perform one or more methods and/or algorithms.

Any of the herein described methods, programs, algorithms or codes may be contained on one or more machine-readable media or memory. The term “memory” may include a mechanism that provides (for example, stores and/or transmits) information in a form readable by a machine such a processor, computer, or a digital processing device. For example, a memory may include a read only memory (ROM), random access memory (RAM), magnetic disk storage media, optical storage media, flash memory devices, or any other volatile or non-volatile memory storage device. Code or instructions contained thereon can be represented by carrier wave signals, infrared signals, digital signals, and by other like signals.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure.

The phrases “in an embodiment,” “in embodiments,” “in various embodiments,” “in some embodiments,” or “in other embodiments” may each refer to one or more of the same and/or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

It should be understood that the description herein is only illustrative of the present disclosure. Various alternatives and modifications can be devised by those skilled in the art without departing from the disclosure. Accordingly, the present disclosure is intended to embrace all such alternatives, modifications and variances. The embodiments described are presented only to demonstrate certain examples of the disclosure. Other elements, steps, methods, and techniques that are insubstantially different from those described above and/or in the appended claims are also intended to be within the scope of the present disclosure.

What is claimed:

1. A computer implemented method for determining an order of nucleotides of an RNA molecule, wherein the method includes:
receiving liquid chromatography-mass-spectrometry (LC-MS) data of an RNA sample, the LC-MS data including a mass, retention time (RT), volume, and quality score (QS);
filtering the LC-MS data based on mass, the filtering including removing masses smaller than a predetermined size;
analyzing the filtered LC-MS data, to determine a plurality of RNA sequences, analyzing the filtered LC-MS data including:
determining a mass difference between at least two adjacent ladder fragments; and
determining whether the mass difference is equal to at least one of a canonical nucleotide, or a modified nucleotide; and
reading-out an RNA sequence as a sequence read after determining no remaining valid nucleotides in the remaining LC-MS data, the RNA sequence including a sequence order of each identified canonical nucleotide and any identified modified nucleotides.

2. The computer implemented method of claim 1, wherein the method further includes:
determining whether there are any gaps in the sequenced LC-MS data;
determining whether there are any remaining RNA fragments that did not yield a valid nucleotide based on the gaps;
performing a hierarchical clustering algorithm on the RNA fragments to identify possible nucleotides from their related mass-adducts, the hierarchical clustering algorithm including:
determining a distance metric based on a mass as well as RT for the compound; and
grouping RNA fragments, into a cluster of masses, based on their mass relationship, such that each fragment includes possible mass-adducts of a true ladder fragment;
determining the mass of an RNA fragment for each cluster, based on item-wise comparison between the identified mass-adducts and the cluster of masses;
predicting a ladder fragment based on the determined mass for each cluster; and
reading-out an RNA sequence based on the predicted ladder fragment, the RNA sequence including any identified mass-adducts.

3. The computer implemented method of claim 1, wherein a length of the RNA molecule is more than 20 nucleotides.

4. The computer implemented method of claim 1, wherein one or more RNA molecules are present in the RNA sample to be sequenced.

5. The computer implemented method of claim 1, wherein the RNA sample includes a purified RNA sample.

6. The computer implemented method of claim 1, wherein the RNA sample includes a therapeutic RNA molecule.

7. The computer implemented method of claim 1, wherein the RNA sequence is determined by correlation of MS data output with a mass of known ribonucleotides.

8. The computer implemented method of claim 1, the method further including determining a type, location, and quantity of modified ribonucleotides based on correlating mass-spectrometry (MS) data output with a mass of known modified ribonucleotides.

9. The computer implemented method of claim 1, wherein the sequencing of the filtered LC-MS data is based on a unique property of an RNA fragment.

10. The computer implemented method of claim 9, wherein the unique property of the RNA fragment includes at least one of electronic or optical signature signals.

11. A system for determining an order of nucleotides of an RNA molecule, wherein the system includes:
one or more processors; and
one or more memories storing instructions which, when executed by the one or more processors, cause the system to:
receive liquid chromatography-mass-spectrometry (LC-MS) data of an RNA sample, the LC-MS data including a mass, retention time (RT), volume, and quality score (QS);
filter the LC-MS data based on mass, the filtering including removing masses smaller than a predetermined size;
analyze the filtered LC-MS data, to determine a plurality of RNA sequences, analyzing the filtered LC-MS data including:
determining a mass difference between at least two adjacent ladder fragments; and
determining whether the mass difference is equal to at least one of: a canonical nucleotide, or a modified nucleotide; and
reading-out an RNA sequence as a sequence read after determining no remaining valid nucleotides in the remaining LC-MS data, the RNA sequence including a sequence order of each identified canonical nucleotide and any identified modified nucleotides.

12. A computer implemented method for determining an order of nucleotides of an RNA molecule, the method including:
receiving liquid chromatography-mass-spectrometry (LC-MS) data of an RNA sample, the RNA sample including an RNA ladder fragment;
accessing a database including theoretical mass, calculated from chemical formula, of all known ribonucleotides including those with modifications to the base;
performing anchor-based sub-setting on the LC-MS data, the anchor based sub-setting including selecting a data zone;
performing base calling on the subset of LC-MS data to generate a dataset of tuples;
building trajectories linking tuples in the dataset to generate a draft read of the RNA ladder fragment; and
performing a draft read strategy.

13. The computer implemented method of claim 12, wherein the draft read strategy includes scoring based on at least one of read length, average volume, average quality score (QS), or average parts per million (PPM).

14. The computer implemented method of claim 13, wherein PPM is determined as:
$$PPM = \frac{M a s {s}_{experimental} - {Mass}_{theoretical}}{M a s {s}_{theoretical}} \times 1 {0}^{6} ,$$
wherein:
Massₑₓₚₑᵣᵢₘₑₙₜₐₗ is an experimental mass corresponding to a ladder fragment including a molecular tag; and
Mass_{theoretical} is the theoretical mass.

15. The computer implemented method of claim 12, wherein average PPM is a sum of all PPM values associated with data points contained in a draft read divided by read length.

16. The computer implemented method of claim 12, wherein building trajectories further includes performing a Depth First Search (DFS) algorithm to ensure that all possible draft reads will be found from the LC-MS data.

17. The computer implemented method of claim 12, wherein the computer implemented method further includes biochemical labeling of the RNA sample.

18. The computational method of claim 12, wherein the draft read strategy includes a global hierarchy ranking strategy or a local best strategy.

19. The computer implemented method of claim 12, wherein the draft read strategy includes a local best strategy.

20. The computer implemented method of claim 12, the method further including performing an alignment/assembly algorithm configured to assemble a complete RNA sequence from different fragments of the RNA molecule.
